(19) United States
(12) Patent Application Publication
(10) Pub. No.: US 2014/0073909 A1
(43) Pub. Date: Mar. 13, 2014

(54) MR Patient Couch with Integrated RF Devices

(71) Applicant: Patrick Gross, Buckenhof (DE)

(72) Inventor: Patrick Gross, Buckenhof (DE)

(21) Appl. No.: 14/022,079

(22) Filed: Sep. 9, 2013

(30) Foreign Application Priority Data
Sep. 10, 2012 (DE) DE 102012216007.8

(51) Int. Cl. A61B 5/055

(52) U.S. Cl. CPC A61B 5/0555
USPC 600/422

(57) ABSTRACT

A patient couch for a magnetic resonance tomography system includes a radio frequency (RF) transmit system and/or an RF receive system provided in the patient couch for at least one local coil. A local coil may be connected to the RF transmit system and/or the RF receive system of the patient couch.

This application claims the benefit of DE 10 2012 216 007.8, filed on Sep. 10, 2012, which is hereby incorporated by reference in its entirety.

### BACKGROUND

The present embodiments relate to a patient couch and a magnetic resonance tomography (MRT) device.

Magnetic resonance devices (MRTs) for examining objects or patients using magnetic resonance tomography are known, for example, from DE10314215B4.

### SUMMARY

The scope of the present invention is defined solely by the appended claims and is not affected to any degree by the statements within this summary.

The present embodiments may obviate one or more of the drawbacks or limitations in the related art. For example, a magnetic resonance tomography (MRT) patient couch is optimized.

### BRIEF DESCRIPTION OF THE DRAWINGS

FIG. 1 shows a schematic representation of a magnetic resonance tomography (MRT) system having one embodiment of a patient couch.

### DETAILED DESCRIPTION

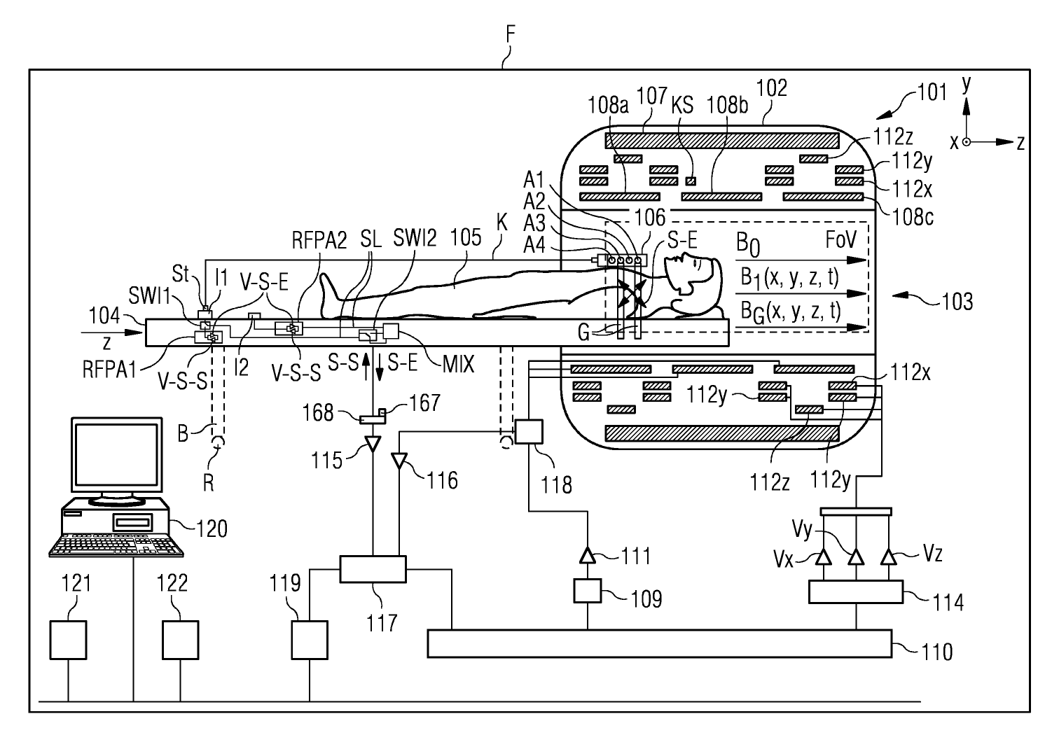

FIG. 1 shows one embodiment of an imaging magnetic resonance device MRT 101 (e.g., located in a shielded room or Faraday cage F) having a whole body coil 102 with, for example, a tubular space 103 into which a patient couch 104 (e.g., a patient table; with supports B and rollers R indicated with a dashed line) with an examination object 105 (e.g., body of a patient; with one or more local coil arrangements 106) may be moved in the direction of the arrow z in order to generate recordings of the patient 105 using an imaging method. One or more local coil arrangements 106 are arranged, for example, on the patient, with which recordings of a subarea of the body 105 may be generated in a local area (e.g., field of view (FOV)) of the MRT 101. Signals of the local coil arrangement 106 may be evaluated (e.g., converted into images, stored or displayed) by an evaluation device (e.g., including elements 168, 115, 117, 119, 120, 121) of the MRT 101. The evaluation device may be connected to the local coil arrangement 106 via coaxial cables or radio (e.g., element 167).

In order to examine a body 105 (e.g., an examination object or a patient) with a magnetic resonance device MRT 101 using magnetic resonance imaging, different magnetic fields attuned to one another as accurately as possible in terms of temporal and spatial characteristic are irradiated onto the body 105. A strong magnet (e.g., a superconducting cryomagnet 107) in a measuring cabin with, for example, a tubular opening 103 generates a statically strong main magnetic field B₀ that amounts, for example, to 0.2 Tesla to 3 Tesla or even more. FIG. 1 is not true to scale (e.g., the superconducting magnet may be thicker). A body 105 to be examined, mounted on a patient couch 104, is moved into an area of the main magnetic field B₀ that is approximately homogeneous in the observation area FoV. Excitation of the nuclear spin of atomic nuclei of the body 105 takes place via magnetic high frequency excitation pulses B1 (x, y, z, t) that are irradiated via a local coil and/or via a body coil 108 (e.g., a multipart body coil 108a, 108b, 108c) as a high frequency antenna (and/or if necessary, a local coil arrangement) that is shown in a very simplified view. High frequency excitation pulses are generated by a pulse generation unit 109, for example, which is controlled by a pulse sequence control unit 110. After amplification with one or more high frequency amplifiers 111 (e.g., RF amplifiers or HF amplifiers), the high frequency excitation pulses are routed to the high frequency antenna 108. The high frequency system shown is only indicated schematically. In other embodiments, more than one pulse generation unit 109, more than one high frequency amplifier 111 and a plurality of high frequency antennas 108 a, b, c are used in a magnetic resonance device 101.

The magnetic resonance device 101 has gradient coils 112x, 112 y, 112 z, with which, during a measurement, magnetic gradient fields B_{G} (x, y, z, t) are irradiated for selective slice excitation and local encoding of the measuring signal. The gradient coils 112x, 112y, 112z are controlled by a gradient coil control unit 114 (and if necessary, via amplifiers Vx, Vy, Vz), which, similarly to the pulse generation unit 109, is connected to the pulse sequence control unit 110.

Signals emitted from the excited nuclear spins of the atomic nuclei in the examination object are received by the body coil 108 and/or at least one local coil arrangement 106, amplified by assigned high frequency preamplifiers 116 and further processed and digitized by a receive unit 117. The recorded measured data is digitized and stored as complex numerical values in a k-space matrix. An associated MR image may be reconstructed from the k-space matrix populated with values using a multi-dimensional Fourier transformation.

For a coil that may be operated both in the transmit and also in the receive mode, such as, for example, the body coil 108 or a local coil 106, the correct signal forwarding is controlled by an upstream transmit-receive switch 118.

An image processing unit 119 generates an image from the measured data. The image is shown to a user via a control console 120 and/or is stored in a storage unit 121. A central computer unit 122 controls the individual system components.

In MR tomography, images with a high signal-to-noise ratio (SNR) may be recorded using local coil arrangements (e.g., coils, local coils). The local coil arrangements are antenna systems that are attached in the immediate vicinity at (anterior) or below (posterior) or on or in the body 105. With an MR measurement, the excited nuclei induce a voltage in the individual antennas of the local coil. The induced voltage is amplified with a low-noise preampflier (e.g., LNA, Preamp) and is forwarded to the receive electronics. High field systems (e.g., 1.5 T-12 T or more) are used to improve the signal-to-noise ratio even in highly resolved images. If more individual antennas may be connected to an MR receive system than there are receivers present, a switching matrix (e.g., RCCS) is integrated between receive antennas and receiver, for example. This routes the currently active receive channels (e.g., the receive channels that lie precisely in the field of view of the magnet) to the existing receivers. More coil elements than there are receivers present may be connected, since with a whole body coverage, only the coils that are disposed in the FoV and/or in the homogeneity volume of the magnet are to be read out.

An antenna system that may include, for example, one or, in the case of an array coil, a number of antenna elements (e.g., coil elements) may be referred to as a local coil arrangement 106, for example. These individual antenna elements are embodied, for example, as loop antennas (loops), butterfly, flex coils or saddle coils, for example. A local coil arrangement includes, for example, coil elements, a preamplifier, further electronics (e.g., decoupling coils), a housing, contacts and may include a cable with a plug, by which the local coil arrangement is connected to the MRT system 101. A receiver 168 attached on the system side filters and digitizes a signal received from a local coil 106 by radio, for example, and transfers the data to a digital signal processing device that may derive an image or a spectrum from the data obtained by a measurement and makes the image or the spectrum available to the user, for example, for subsequent diagnosis by the user and/or for storage purposes.

FIG. 1 also shows an exemplary embodiment of a patient couch 104 for a magnetic resonance tomography system 101. The patient couch 104 has at least one RF transmit system (RFPA1, RFPA2, MIX, V-S-S) and/or at least one RF receive system (RFPA1, RFPA2, MIX, V-S-E) for at least one local coil. At least one local coil 106 (e.g., with a socket or plug St) may be connected to the RF transmit system and/or the RF receive system of the patient couch 104 via, for example, one of a plurality of interfaces I1, I2 (e.g., with a plug or socket).

When developing an MRT system, transmission paths (e.g., radio-frequency paths) are to be provided for HF signals (S-S) to be sent to an examination object 105 and/or HF signals (e.g., RF signals) to be received from an examination object. The transmission paths are flexible.

For “multi-nuclear-spectroscopy,” an MR system uses room intended for upgrades (e.g., further developments) to the scanner and links between the transmit system and the MR couch. Future options and capacities may be provided in current platforms in multi-nuclear MRT transmit paths.

One embodiment of an MRT system with, for example, a number of independent local coils A1-A4 of one or a plurality of local coils is provided. The MRT system is configured to be accordingly flexible in terms of infrastructure.

Excitation of the nuclear spins of atomic nuclei of the body 105 takes place via magnetic (RF-/HF) high frequency excitation pulses B1 (x, y, z, t) that are also irradiated, for example, via at least one high frequency antenna A1-A4 of a local coil arrangement 106 (shown simplified) (e.g., local coil with an HF antenna array, head coil, shoulder coil, foot coil).

High frequency excitation pulses B1 (x, y, z, t) are generated, for example, by a pulse generation unit 109 that is controlled by a pulse sequence control unit 110 and generates HF excitation signals S-S to be sent. After amplification of the HF excitation signals S-S to be sent using RF amplifiers 116, RFPA1, RFPA2, V-S-S (e.g., of which all or some may be integrated in the patient couch 104), the HF excitation signals S-S are routed to one or more high frequency antennas A1, A2, A3, A4 (e.g., two, more than two, more than eight, more than sixteen, more than thirty-two, more than sixty-four or more than ninety-six) in a local coil 106 or in a plurality of local coils and sent as high frequency excitation pulses B1 (x, y, z, t).

Signals S-E emitted from the examination object 105 may also (e.g., only or in addition to sending) be received by the local coil 106 and/or amplified (RF-PA1, RF-PA2) and/or mixed with a mixer (MIX).

According to one embodiment, RF transmit systems are provided for one or a plurality of local coils 106 in a patient couch 104 that may be used with an MRT 101 (e.g., may be connected or mechanically docked). The signal lines SL of the local coils 106 (e.g., in sections) may also run in the patient couch 104. The RF transmit systems and/or the RF receive systems (e.g., RF-PA1, RFPA2, mixers, switching facilities SW1, SW2) may also be arranged at least partially in the patient couch 104.

Receive systems for HF signals received from an examination object 105 may be provided entirely or partially in the patient couch (e.g., one or a plurality of HF amplifiers of a receive system, mixers of a receive system).

An RF transmit system (e.g., RFPA1, RFPA2, MIX, V-S-S) and/or an RF receive system (e.g., RFPA1, RFPA2, MIX, V-S-E) optionally arranged (e.g., optionally switchable) in the patient couch 104 may optionally be implemented in this respect. For example, a switching device SWI1 such as a toggle SWI1 that may be actuated locally or controlled remotely by the MRT 101 may be provided. Depending on the switch setting of which a signal is routed to (S-S) and/or from (S-R) the local coil 106 directly (e.g., not via the amplification device) or via an amplification device RFPA1 (e.g., with, if applicable, amplifiers V-S-S for signals S-S to be sent and/or with, if applicable, amplifiers V-S-E for received signals S-E).

For example, a mixer MIX of an RF transmit system (RFPA1, RFPA2, MIX, V-S-S) and/or RF receive system (RFPA1, RFPA2, MIX, V-S-E) optionally arranged (e.g., optionally switchable) in the patient couch 104 may optionally also be implemented in this respect, for example, as a switching device SWI2. For example, a toggle SWI2, which may be actuated locally or controlled remotely by the MRT, may be provided, depending on the switch setting of which signals are routed to (S-S) and/or from (S-R) the local coil 106 directly (e.g., not via the mixer) or via the mixer MIX.

In this way, a relatively generic infrastructure for the patient couch may be provided (e.g., a power supply, control signals, bus signals, clocks and potentially different pre-stresses). Prerequisites for MKO may also be implemented in the patient couch.

Advantages of such embodiments may be, for example, fewer infrastructure demands on standard systems, improved design-cost ratios and more diverse application possibilities in the future, modular upgrade (e.g., further development) possibilities in the field and/or at the site of the assembled MRT and service using switchable couches (e.g., according to requirements by local coils), and the possibility of a comprehensive and/or dedicated solution, also by integrating special coils in the patient couch.

The power for a local transmit or transmit of, for example, HF signals via at least one local coil and, for example, for comparatively low frequency atomic nuclei (e.g., low frequency nuclei) may be relatively low. Current RF power amplifiers allow MRT systems to be equipped also with RF amplifiers at the bore 103 of an MRT. These allow for integration within a patient couch, which may assist with special adjustments in a modular sense.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A patient couch for a magnetic resonance tomography system, the patient couch comprising;
a radio frequency (RF) transmit system, an RF receive system, or an RF transmit and receive system provided in the patient couch, a local coil being connectable to the RF transmit system, the RF receive system, or the RF transmit and receive system.

2. The patient couch as claimed in claim 1, comprising the RF transmit system, a plurality of local coils being connectable to the RF transmit system, the plurality of local coils comprising the local coil.

3. The patient couch as claimed in claim 1, further comprising one or more RF amplifiers provided in the patient couch,
wherein the patient couch is configured to transmit, via the one or more RF amplifiers, signals generated by a pulse generation unit to one high frequency antenna or a plurality of high frequency antennas in the local coil or a plurality of local coils, respectively, the local coil being one of the plurality of local coils, the local coil or the plurality of local coils configured to transmit high frequency excitation pulses.

4. The patient couch as claimed in claim 1, further comprising one or more RF amplifiers provided in the patient couch,
wherein the patient couch is configured to transmit, via the one or more RF amplifiers, high frequency excitation pulses generated by a pulse generation unit to one high frequency antenna or a plurality of high frequency antennas in the local coil or a plurality of local coils on the patient couch, respectively, for at least one connection plug or a connection socket of a connection to the local coil, the local coil being one of the plurality of local coils.

5. The patient couch as claimed in claim 4, wherein the patient couch is configured to transmit the high excitation pulses generated by the pulse generation unit via an interface.

6. The patient couch as claimed in claim 1, comprising the RF receive system for the local coil,
wherein the RF receive system comprises a mixer operable to mix signals received from an examined object.

7. The patient couch as claimed in claim 6, wherein the mixer is operable to mix signals received from the examined object via a plurality of high frequency antennas.

8. The patient couch as claimed in claim 1, comprising the RF receive system for the local coil, the RF receive system comprising one or more amplifiers operable to amplify signals received from an examined object.

9. The patient couch as claimed in claim 8, wherein the one or more amplifiers comprise one or more RF amplifiers.

10. The patient couch as claimed in claim 1, wherein an RF transmit system, an RF receive system, or an RF transmit and receive system arranged on the patient couch is switchable or bypassable via a switching device as a function of a switching state of the switching device.

11. The patient couch as claimed in claim 1, wherein a mixer of the RF transmit system, the RF receive system, or the RF transmit and receive system arranged in the patient couch is switchable or bypassable via a switching device as a function of a switching state of the switching device.

12. The patient couch as claimed in claim 1, further comprising a transmit-receive switch arranged in the patient couch.

13. The patient couch as claimed in claim 1, further comprising at least one transmit-receive switch arranged in the patient couch, the transmit-receive switch being switchable, via a switching device as a function of a switching state of the switching device, into a signal path for receive signals, for transmit signals, or for the receive signals and the transmit signals.

14. A magnetic resonance tomography system comprising:
a patient couch comprising:
a radio frequency (RF) transmit system, an RF receive system, or an RF transmit and receive system provided in the patient couch, a local coil being connectable to the RF transmit system, the RF receive system, or the RF transmit and receive system.

15. The magnetic resonance tomography system as claimed in claim 14, comprising the RF transmit system, a plurality of local coils being connectable to the RF transmit system, the local coil being one of the plurality of local coils.

16. The magnetic resonance tomography system as claimed in claim 14, further comprising one or more RF amplifiers provided in the patient couch,
wherein the patient couch is configured to transmit, via the one or more RF amplifiers, signals generated by a pulse generation unit to one high frequency antenna or a plurality of high frequency antennas in the local coil or a plurality of local coils, respectively, the local coil being one of the plurality of local coils, the local coil or the plurality of local coils configured to transmit high frequency excitation pulses.

17. The magnetic resonance tomography system as claimed in claim 14, further comprising one or more RF amplifiers provided in the patient couch,
wherein the patient couch is configured to transmit, via the one or more RF amplifiers, high frequency excitation pulses generated by a pulse generation unit to one high frequency antenna or a plurality of high frequency antennas in the local coil or a plurality of local coils on the patient couch, respectively, for at least one connection plug or a connection socket of a connection to the local coil, the local coil being one of the plurality of local coils.

18. The magnetic resonance tomography system as claimed in claim 17, wherein the patient couch is configured to transmit the high excitation pulses generated by the pulse generation unit via an interface.

19. The magnetic resonance tomography system as claimed in claim 14, comprising the RF receive system for the local coil,
wherein the RF receive system comprises a mixer operable to mix signals received from an examined object.

20. The magnetic resonance tomography system as claimed in claim 19, wherein the mixer is operable to mix signals received from the examined object via a plurality of high frequency antennas.
